(19) United States
(12) Patent Application Publication
(10) Pub. No.: US 2020/0253165 A1
(43) Pub. Date: Aug. 13, 2020

(54) ANIMAL URINE COLLECTION AND MONITORING SYSTEM

(71) Applicants: James David Luciew, Johnstown, PA (US); Michele Lynn Luciew, Johnstown, PA (US)

(72) Inventors: James David Luciew, Johnstown, PA (US); Michele Lynn Luciew, Johnstown, PA (US)

(21) Appl. No.: 16/790,201

(22) Filed: Feb. 13, 2020

Related U.S. Application Data
(60) Provisional application No. 62/805,161, filed on Feb. 13, 2019.

(51) Int. Cl. A01K 23/00; A61F 13/505; A61F 13/74; A61F 13/82

(52) U.S. Cl. CPC A01K 23/00; A61F 13/505; A61F 2013/15186; A61F 13/82; A61F 13/74

(57) ABSTRACT

An animal urine collection and monitoring system embodying a device to collect urine from a male animal and monitor the animal's urinary activities.

### CROSS-REFERENCE TO RELATED APPLICATION

This application claims the benefit of priority of U.S. provisional application No. 62/805,161, filed 13 Feb. 2019, the contents of which are herein incorporated by reference.

### BACKGROUND OF THE INVENTION

The present invention relates to animal housebreaking and, more particularly, to an animal urine collection and training system embodying a device to collect urine from the animal and, in an enhanced embodiment, to monitor and provide instant notification on the animal's urinary activities using integrated and wireless technologies.

Existing animal diapers and wraps simply cover the animal's belly area with absorbent material or cloth similar to a human diaper. And so, after urine is dispensed into the diaper or wrap, it remains in contact with the animal's skin until the diaper is removed. Being in a wet diaper is uncomfortable for the animal and can result in skin irritation, unpleasant odors, and urinary tract infections. What is more, the wrap is not designed to follow the unique contours of the animal's body, resulting in a poor fit that causes discomfort and leaves gaps that can allow urine to escape. In addition, “re-usable” cloth approaches require laundering after use and often sag the more they are loaded. In other words, existing solutions do not include a fitted urine reservoir made from a flexible silicone or soft, moisture repellent skin safe material. Existing products also lack the necessary technology to monitor, notify, and manage the animal's urine activity and, in concert with a companion software application, assist with housebreaking and the ordering of related products and services.

As can be seen, there is a need for an animal urine collection and monitoring system embodying a device to collect urine from an animal and monitor the animal's urinary activities. The systemic device is dimensioned and adapted to keep the animal's body dry while preventing the animal from marking their territory by directing urine into a collection reservoir that can be fitted with disposable, absorbent sanitary pads.

The collection reservoir covers the animal's urinary meatus, also known as the external urethral orifice, and remains fixed in place so that the urine is always directed into the collection reservoir, thereby assisting with animal house breaking and preventing unwanted indoor marking. The collection reservoir provided by the systemic device can be fitted with absorbent sanitary pad inserts.

Furthermore, the contoured fit of the systemic device keeps the collection reservoir in position on the animal while making it comfortable for the animal to wear. Anatomical fit is accomplished with a unique angled strap design in relation to the collection reservoir that, when fitted to the animal, forms a tapered “cone” that perfectly abuts to the animal torso, while it generates upward lift necessary to keep the urine collection reservoir in position on the animal during normal use.

The urine collection reservoir is designed to accommodate the animal's unique anatomical features, forming a comfortable seal to reduce skin-to-urine contact, while preventing urine from leaking out when it is being dispensed. The collection reservoir is adapted to accept disposable, absorbent sanitary pads. The upper circumference of the collection reservoir is fitted with an integrated lip that keeps urine from spilling out during and after discharge, while helping to direct the flow of urine into the collection reservoir and onto any absorbent sanitary pads installed within the collection reservoir. The integrated reservoir lip also keeps the absorbent sanitary pads securely positioned within the collection reservoir, while forming a barrier between the collection reservoir contents and the animal's torso. Thus, as urine is collected and absorbed, the animal remains dry, and contact with urine is minimized. Additionally, embedded sensor electronics add monitoring and push notification features to alert caregivers, speed training, and track animal behavior as the device continuously checks for moisture.

The collection reservoir is designed to fit over and cover the animal's urine discharge area, the urethral orifice, while an integrated lip forms a seal between the collection reservoir and the animal's abdominal cavity. The overhang lip provides an arcuate shape designed to provide a comfortable fit against the abdominal cavity. The integrated lip is dimensioned and adapted to keep urine from spilling out of the reservoir when the animal is lifting its leg. As seen from the side, the top front of the external reservoir is sloped downward to accommodate the animal's chest cavity. As seen from the top, the top front area forms an arcuate shape to maintain a comfortable seal between the reservoir and the animal abdomen. As seen from the rear, the top rear of the collection reservoir is sloped downward, defining a recess to accommodate the animal's penis shaft and reduce pressure. Pressure to the penis area is further dampened by the rounded form and flexible nature of the integrated lip element that follows the perimeter of the collection reservoir.

Urine does not escape the product due to the systemic combination of the angled strapping, contoured collection reservoir, absorbent sanitary pads, and the integrated lip. The angled strapping allows the device to abut against the animal torso, through generating the upward lift necessary to keep the collection reservoir in position on the animal. The contoured nature of the collection reservoir and integrated lip conforms to the animal's anatomy, while also acting as a container to accommodate and direct the flow of urine onto the sanitary pads that are kept in position inside the collection reservoir cavity. Finally, the integrated lip forms a barrier around the circumference of the contoured collection reservoir, preventing the urine from escaping the cavity while it is being absorbed by the sanitary pads, minimizing contact between urine and the animal's skin. The product is made from durable and flexible materials making it easy to clean and safe for the animal to wear.

Through the angled strap arrangement, the systemic device is designed to accommodate the animal's torso, hind legs, and penis, promoting natural motion and making it comfortable for the animal to wear while remaining in the correct position relative to the animal's urethral orifice or penis. This is accomplished by a deliberate angled arraignment of the reservoir relative to the band/strap elements. When viewed from the top or bottom while the straps are released and in an open condition, the straps are set at a distinct angle in relation to the remote reservoir. When fitted to the animal, the angle of the straps in relation to the remote reservoir form a cone that fits the animal torso, while creating lift necessary to keep the front of the reservoir in constant contact with the animal torso and in position over the urethral orifice. Depending on animal size, the measurement of the angle may differ to best accommodate the differing size of the animals abdominal and torso. In any case, the angled orientation of the straps in relation to the remote reservoir is clearly visible and advantageous to the invention. By angling the straps, the front (animal chest facing) edge of the band is dimensionally longer than the rear (animal tail facing) edge, thereby creating a larger circumference along the front (animal chest facing) opening and a smaller circumference along the rear (animal tail facing) opening while the device is fastened and in a closed position on the animal. Thus, when the device is fastened and fitted to the animal, the resulting shape forms a tapered cone that minimizes gaps along the tail facing edge, produces more upward lift to the front of the device, and comfortably adheres to the cone shaped nature of the animal's torso.

Further, the straps are flexible and adjustable sufficient to secure the fitted urine collection reservoir to the animal. The collection reservoir is made from an easy to clean and skin safe material such as silicone. The collection reservoir is designed to cover the animal's urine discharge area, the urethral orifice, while accommodating the animal's hind legs to allow for normal and natural movement.

In certain embodiments, the collection reservoir can also be fitted with a separate cap that acts as a barrier between the animal and the collection reservoir. The cap acts as a lid that allows urine to flow into the reservoir cavity while keeping the animal's skin dry. The collection reservoir may or may not be fitted with absorbent material or inserts that help gather and collect the urine.

Embedded sensor technology and electronic components add monitoring, notification and ordering features via a companion software application. The software application may be adapted to aid in ordering refill absorbent insert pad, in monitoring and tracking animal urinary activity, in assisting with house training the animal, and in assisting with overall pet health. Specifically, the device may provide an optional sensor to track animal urine activity and notify the animal owner when the urine reservoir is to be emptied and refill absorbent pads added.

### SUMMARY OF THE INVENTION

In one aspect of the present invention, a device for collecting urine from an animal includes the following: a reservoir assembly having a body defining a urine reservoir; the body providing: two opposing side portions extending to a first elevation and sloping downwardly to a front portion to accommodate a chest cavity of the animal; the front portion having an arcuate shape defined by a front edge along a second elevation; a rear portion depending from the two opposing side portions so that an associated rear edge defines a U-shape extending to a third elevation; and the second elevation disposed between the first elevation and the third elevation; an overhang lip extend inward from the two opposing side portion, the front edge, and the rear edge so as to define a continuous periphery of an opening communicating to the urine reservoir; and two straps extending from the two opposing side portions at an acute angle of incidence relative to a longitudinal line extending between the two opposing side portions wherein the acute angle of incidence is between ten and fifty degrees relative to the longitudinal line, and wherein the front edge extends for a length longer than a length of the rear edge.

In another aspect of the present invention, the device for collecting urine from an animal may include an absorbent pad dimensioned to be received along a bottom of the urine reservoir.

In yet another aspect of the present invention, the device for collecting urine from an animal may include the following: at least one moisture sensor operatively associated with the urine reservoir; and a microprocessor electrically connected to at least one moisture sensor, the microprocessor configured to signaling a remote computing device when the moisture sensor detects a predetermined amount of moisture.

In yet another aspect of the present invention, a method of kenneling an animal in a confined space or home with no access to an outdoor environment includes the following: providing the above-mentioned device for collecting urine from an animal; placing at least one absorbent pad in the bottom of the urine reservoir; wrapping the two straps around the waist of the animal so that urine reservoir circumscribes a urethral orifice of the animal; and in the male embodiment, abutting the associated rear edge along a penile shaft of the animal; and monitoring the remote computing device or using artificial intelligence for a determination of when to dispatch caregivers to replace the absorbent pad.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a rear perspective view of an exemplary embodiment of the present invention, shown in an attached arrangement;

FIG. 3 is a top plan view of an exemplary embodiment of the present invention, illustrating two straps 16A and 16B extending from the two opposing side portions at an acute angle of incidence 50 relative to a longitudinal line extending between the two opposing side portions 52, a urine collection reservoir 18 and an operatively associating removable/insertable absorbent pad 28;

FIG. 4 is a partial section view of an exemplary embodiment of the present invention, taken along line 4-4 of FIG. 3;

FIG. 5 is an elevation view of an exemplary embodiment of the present invention, shown in the attached arrangement;

FIG. 6 is a front elevation view of an exemplary embodiment of the present invention, shown in the attached arrangement;

FIG. 7 is a rear elevation view of an exemplary embodiment of the present invention, shown in the attached arrangement;

FIG. 8 is an operational flow chart of an exemplary embodiment of the present invention; and

FIG. 9 is a schematic view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an animal urine collection and monitoring system embodying a device to collect urine from an animal and monitor the animal's urinary activities.

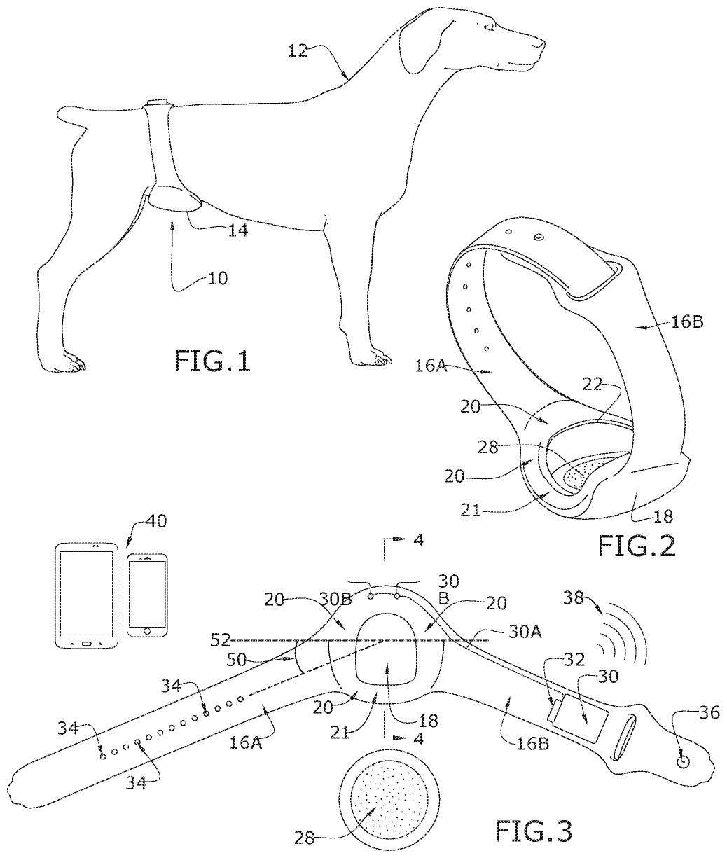

It should be understood by those skilled in the art that the use of directional terms such as upper, lower, upward, downwardly, top, rear (rearward), front and the like are used in relation to the illustrative embodiments as they are depicted in the figures, the upward direction (or upper) being toward the top of the corresponding figures, downward (lower) direction being toward the bottom of the corresponding figures, and the rearward direction being defined in relationship of the animal shown in FIG. 1 of the corresponding figures.

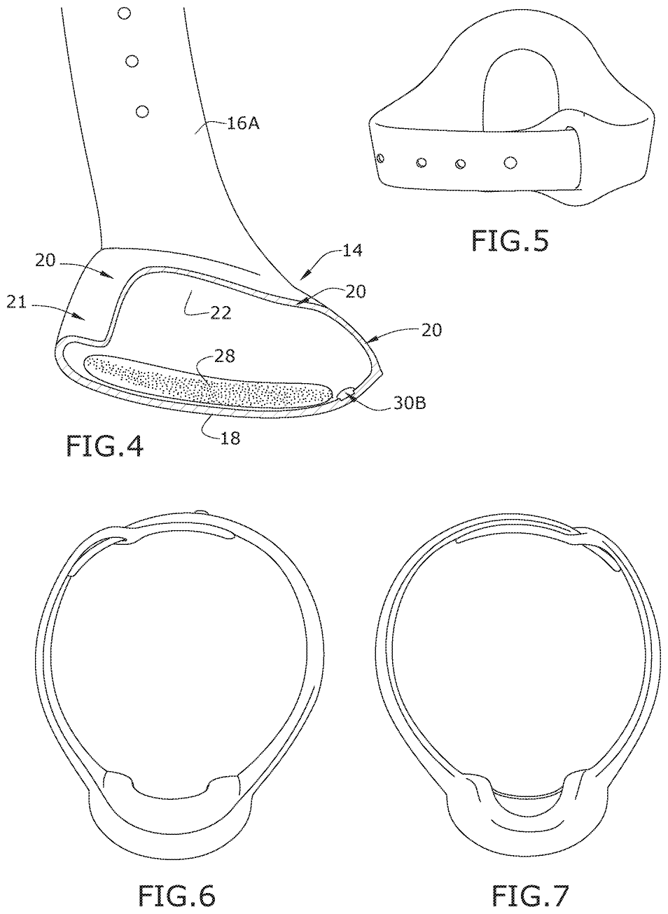

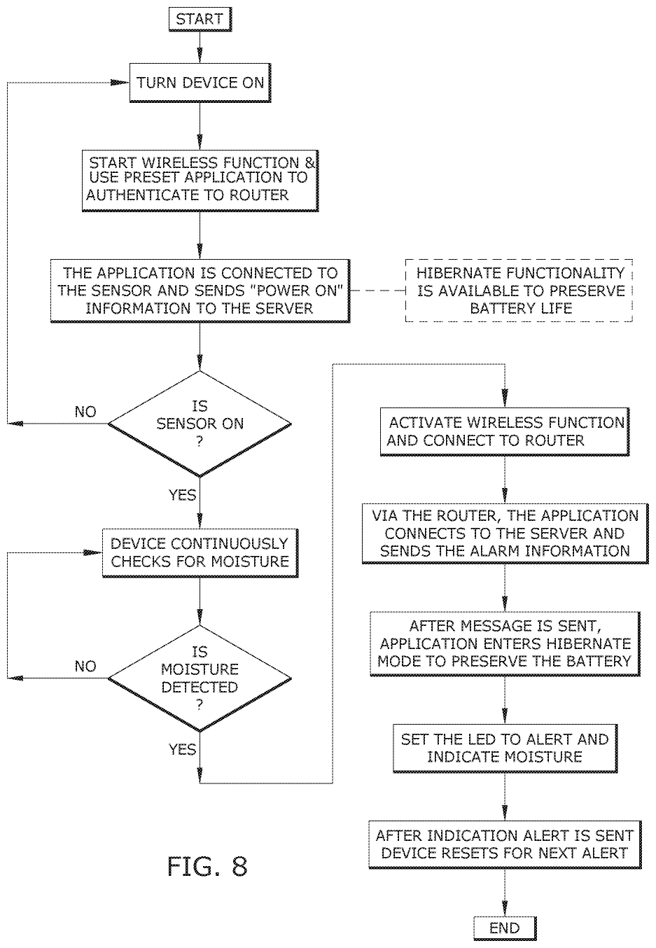

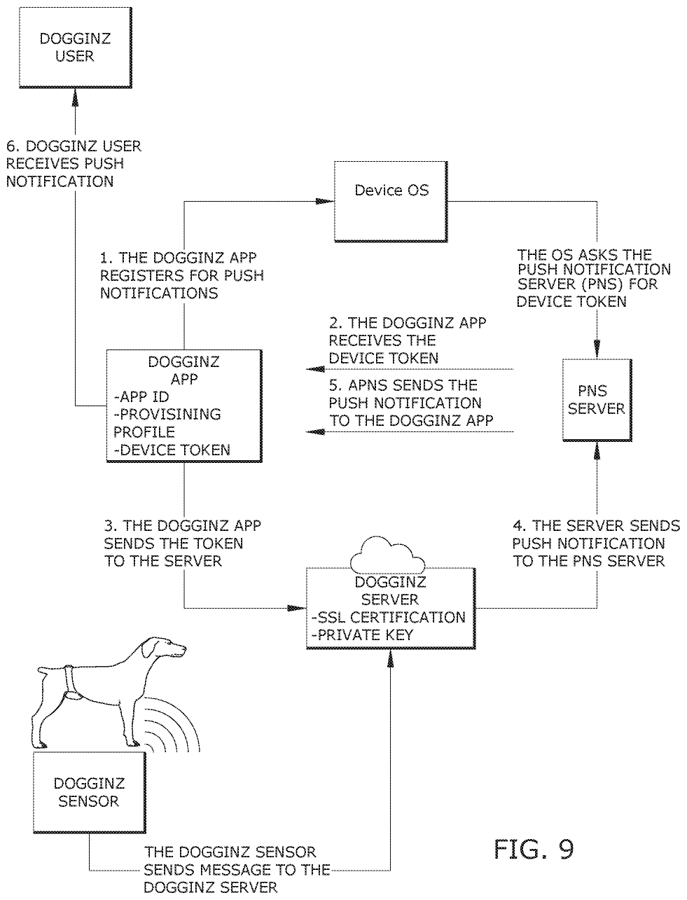

Referring to FIGS. 1 through 9, the present invention may include an animal urine collection and monitoring system embodying a systemic device 10 to collect urine from the urethral orifice of an animal 12 and monitor the urinary activities of said animal 12. The systemic device 10 may include a reservoir assembly 14 connected between two elastic adjustable straps 16A and 16B.

The reservoir assembly 14 may include a urine collection reservoir 18 dimensioned and adapted to circumscribe the urethral orifice of the animal (for a male animal, the penis of the animal 12) and house an absorbent, removable pad 28, resulting in absorbency performance similar to larger disposable diaper products. The urine collection reservoir 18 is constructed from a flexible silicone, rubber, or similar animal skin-safe material. The urine collection reservoir 18 is flexible and conforms to area the animal surrounding the urethral orifice. Along an upper portion of the reservoir assembly 14 may be an integrated lip 20 defining a periphery 22 of an opening that communicates to the urine reservoir 18. The integrated lip 20 overhangs a portion of the collection reservoir 18 so as to abut the skin of the animal 12 when the systemic device 10 is in a worn condition, as illustrated in FIG. 1.

The integrated lip 20 is dimensioned and adapted to keep dispersed urine directed downwardly, collected urine from spilling out, and the absorbent removable pad 28 secured, while the integrated lip 20 engages the animal skin. The integrated lip 20 acts as a barrier between the animal's skin and the urine reservoir to keep the animal dry, while also directing dispersed urine into the urine collection reservoir 18. The integrated lip 20 is constructed from a soft flexible, and easy to clean silicone or similar skin safe material. The integrated lip 20 may provide an animal penis shaft recess 21 to accommodate the penis of a male animal. In certain embodiments, the integrated lip 20 may be removably connected to the reservoir assembly 14 as a separate cap that fits over the reservoir opening.

The two straps 16A and 16B may be integrated into the product or attached separately. In the case where the flexible straps 16A and 16B are integrated with the urine reservoir, it may be cast from the same material as the urine reservoir. One of the straps (16A) may provide adjustment holes 34 along its length for selectively and removably engaging the two straps 16A and 16B in an attached arrangement, as illustrated in FIG. 2.

Two straps 16A and 16B are set at an angle 52 relative to the collection reservoir assembly 14 to enable the systemic device 10 to adhere/abut to the animal torso without any gaps or slippage. The angled straps 16A and 16B are adapted to elevate the urine reservoir 18 keeping it in contact with the animal abdominal wall; in other words, the tapered nature of the angled straps 16A and 16B when fitted to the animal, adhere vertically in a slight rearward angle on the animal, to generate upward lift on the front of the reservoir assembly 14. When equipped, the angle 52 of the two straps 16A and 16B relative to the collection reservoir assembly 14 form a cone that conforms to the tapering nature of the animal torso 12 that is wider at the chest and narrower at the waist.

When viewed from the top or bottom, when in an open condition as illustrated in FIG. 3, the two straps 16A and 16B are set at an angle in relation to the remote reservoir; specifically, the two straps 16A and 16B have an acute angle of incidence 50 relative to a plane 52 that generally bisects the reservoir assembly 14, as illustrated in FIG. 3, and as such the two straps 16A and 16B have an acute angle of incidence relative to the reservoir assembly 14. Further, the two straps 16A and 16B angle away from collection reservoir 18 in a rearward direction, similar to the relationship between wings and fuselage of a common passenger plane. The relationship between the straps 16A and 16B and remote urine reservoir 18 form an acute angle—i.e., an angle of incidence between ten and fifty degrees. It is possible for someone to argue that the bands are set at an obtuse angle when measured from the center and front. Depending on animal size, the degree of the angle will differ to best accommodate the transition cone between the animals abdominal and torso. By angling the two straps 16A and 16B in this way, the front edge is longer than the rear edge, creating a tapered effect that mirrors a deep-chested torso of the animal 12 when the systemic device 10 is fastened. In any case, the angled nature of the bands in relation to the remote urine reservoir 18 is clearly visible and advantageous to the present invention. The two straps 16A and 16B can be made from elastic, cloth, leather, silicone, rubber, or any similar material. In all embodiments, the two straps 16A and 16B are set at an angle in relation to the remote reservoir; to adapt the systemic device 10 to the animal 12, generate upward lift on the front of the reservoir assembly 14, ultimately securing the remote urine reservoir 18 over the animal's abdominal wall; and along the animal's torso 12 with minimal gaps.

The systemic device 10 may be fitted with optional electronic components and sensors to monitor animal urinary activity via Wireless technology. A corresponding software application may be associated with the electronic components and sensors to facilitate sensor operations, for the purpose of tracking animal activity and sending push notifications to a user's computing device 40 when the animal 12 urinates; thereby assisting in house breaking training, reordering of absorbent pads 28 and dispatch of caregiving services. The software application may be adapted to help monitor animal activity for house breaking or house training the animal 12.

The electronic components may include a sensor microcontroller PCB 30 or equivalent, sensor nodes for detecting moisture, associated with a wireless component 32 for receiving and transmitting wireless signals 38 to the computing device 40 and a flexible wire 30A that articulates and stretches within the systemic device 10. The sensor may include urine or moisture sensors electrically connected to sensor wires 30A and sensor nodes 30B. The computing device 40 may provide a user interface that displays all animal activity captured by the sensors.

Sensor hardware is assigned a unique device ID at the factory. A cloud server with specialized software manages communications and continuously listens for all connections from the sensor hardware, based on the unique hardware ID. When data is received from a registered hardware device, the message is sent to the push notification server. The cloud server also receives requests sent by the systemic software application and responds. The communication data of each communication is logged to the server database.

When moisture is detected, the integrated wireless function is activated and connects to the users router, sending notification information to the corresponding server. After the notification is sent, the wireless function of the hardware is hibernated to preserve battery and the hardware LED is set to indicate a moisture alert. Meanwhile, the server queries the token of the registered device, according to the device ID of the hardware, and sends a push notification to the corresponding device through the push notification server.

The sensor hardware is bound to the user and configured to access their desired network with a companion software setup application. The companion software application registers the unique hardware ID for each assigned sensor with the push notification server and the cloud server. The corresponding software allows for the efficient setup of the sensor hardware, subscribing and binding of the unique device ID to the user and simplified connection to the users wireless network. The companion software and server technology permits users to monitor an unlimited number of registered sensor hardware devices, thereby making it a suitable platform for kennel management, efficient dispatch of caregivers, and other related offerings.

A method of using the present invention may include the following. The systemic device 10 disclosed above may be provided. A user may insert an absorbent pad 28 into remote urine reservoir 18. Then the user may wrap the systemic device 10 around a waist of the animal 12 so that its penis is received in the shaft recess 21 of the reservoir assembly 14 and its urethral orifice is circumscribed by the collection reservoir 18. Then the two straps 16A and 16B may be moved to the attached arrangement around said waist and removably secured by way of a locking device 36. While being worn by the animal, the systemic device 10 may collect the discharged urine of the animal and provide the user with monitoring and tracking data by way of the user's computing device 40. In a similar manner, the sensor and notification technology can be leveraged for human use, specifically in personal care and skilled care settings where large numbers of patients will benefit from having a urine sensing device to notify and alert caregivers, thus preventing harmful infections and skin diseases.

It should be understood, of course, that the foregoing relates to exemplary embodiments of the invention and that modifications may be made without departing from the spirit and scope of the invention as set forth in the following claims.

What is claimed is:

1. A device for collecting urine from an animal, comprising:
a reservoir assembly having a body defining a urine reservoir;
the body comprising:
two opposing side portions extending to a first elevation and sloping downwardly to a front portion for accommodate a chest cavity of the animal;
the front portion having an arcuate shape defined by a front edge along a second elevation;
a rear portion depending from the two opposing side portions so that an associated rear edge defines a U-shape extending to a third elevation; and
the second elevation disposed between the first elevation and the third elevation;
an overhang lip extends inward from the two opposing side portion, the front edge, and the rear edge so as to define a continuous periphery of an opening communicating to the urine reservoir; and
two straps extending from the two opposing side portions at an acute angle of incidence relative to a longitudinal line extending between the two opposing side portions.

2. The device for collecting urine from an animal of claim 1, further comprising:
an absorbent pad dimensioned to be received along a bottom of the urine reservoir.

3. The device for collecting urine from an animal of claim 1, wherein the acute angle of incidence is between ten and fifty degrees relative to the longitudinal line.

4. The device for collecting urine from an animal of claim 1, wherein the front edge extends for a length longer than a length of the rear edge.

5. The device for collecting urine from an animal of claim 1, further comprising at least one moisture sensor operatively associated with the urine reservoir.

6. The device for collecting urine from an animal of claim 5, further comprising a microprocessor electrically connected to at least one moisture sensor, configured to detect a predetermined amount of moisture.

7. The device for collecting urine from an animal of claim 5, further comprising a wireless module that wirelessly transmits signals to a cloud server over available wireless or cellular networks without need for additional hardware or base stations.

8. A method of kenneling an animal in a confined space with no access to an outdoor environment, comprising:
providing the device for collecting urine from an animal of claim 6;
placing at least one absorbent pad in the bottom of the urine reservoir;
wrapping the two straps around a waist of the animal so that urine reservoir circumscribes a urethral orifice of the animal; and
configuring the sensor hardware to communicate with the users computing device.

9. The method of claim 8, further comprising:
configuring the sensor hardware to wirelessly transmit signals to a cloud server over available wireless or cellular networks without need for additional hardware or base stations;
assigning notifications to specified caregivers with the users computing device; and
monitoring sensor activity with a remote computing device for a determination of when to dispatch caregivers and replace absorbent pads.
